(19) United States
(12) Patent Application Publication
(10) Pub. No.: US 2024/0177404 A1
(43) Pub. Date: May 30, 2024

(54) HAND-HELD MAKEUP APPLICATOR WITH SENSORS TO SCAN FACIAL FEATURES

(71) Applicant: L'Oreal, Paris (FR)

(72) Inventors: Juwan Hong, Jersey City, NJ (US); Fred Orsita, Wayne, NJ (US); Maya Kelley, San Francisco, CA (US)

(73) Assignee: L'Oreal, Paris (FR)

(21) Appl. No.: 18/517,851

(22) Filed: Nov. 22, 2023

Related U.S. Application Data
(60) Provisional application No. 63/385,468, filed on Nov. 30, 2022.

(30) Foreign Application Priority Data
Feb. 24, 2023 (FR) 2301689

(51) Int. Cl. G06T 15/20; A45D 44/00; G06T 17/00; G06V 10/141; G06V 10/60; G06V 40/16; G06V 40/60

(52) U.S. Cl. CPC G06T 15/205; A45D 44/005; G06T 17/00; G06V 10/141; G06V 10/60; G06V 40/161; G06V 40/171; G06V 40/67; A45D 2044/007

(57) ABSTRACT

A scanning device, including a body, a scanner coupled with the body, a position sensor coupled to the scanner, a camera configured to take a plurality of images as the scanner moves over a facial feature, and a processor. The processor may be configured to detect a position and a curvature of the facial feature based on the position sensor, combine the plurality of images into a single image, and generate a three-dimensional model of the facial feature. Further, a method including moving a scanning device over a facial feature, taking a plurality of images of the facial feature with a camera as the scanning device moves over the facial feature, detecting a position of the facial feature with the position sensor as the scanning device moves over the facial feature, combining the plurality of images together, and generating a three-dimensional model of the facial feature.

### SUMMARY

In one aspect, a scanning device, is disclosed, including a body; a scanner coupled with the body, a position sensor coupled to the scanner, a camera configured to take a plurality of images as the scanner moves over a facial feature; and a processor configured to detect a position and a curvature of the facial feature based on the position sensor, combine the plurality of images into a single image, and generate a three-dimensional model of the facial feature.

In another aspect, a method including moving a scanning device as described herein over a facial feature, taking a plurality of images of the facial feature with a camera as the scanning device moves over the facial feature, detecting a position of the facial feature with the position sensor as the scanning device moves over the facial feature, combining the plurality of images together, and generating a three-dimensional model of the facial feature is disclosed.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1A is a perspective front-side of an example scanning device, in accordance with the present technology;

FIG. 1B is a perspective back-side of the example scanning device of FIG. 1A, in accordance with the present technology;

FIG. 2A is an exploded backside view of an example scanning device, in accordance with the present technology;

FIG. 2B is an exploded frontside view of the example scanning device of FIG. 1A, in accordance with the present technology;

FIG. 3A is an image of an example scanning device in use, in accordance with the present technology;

FIG. 3B is an example three-dimensional model generated by a scanning device, in accordance with the present technology;

FIG. 4 is a user interface displaying a scanning guide, in accordance with the present technology;

FIG. 5 is an example method of using a scanning device, in accordance with the present technology;

FIG. 6 is an example method of using a scanning device having a light source, in accordance with the present technology; and

FIG. 7 is an example method of using a scanning device with a scanning guide, in accordance with the present technology.

### DETAILED DESCRIPTION

Disclosed herein is a scanning device and related methods of using the scanning device to generate a three-dimensional model of a facial feature. In some embodiments, the three-dimensional model is used to fabricate a makeup overlay.

In one aspect, a scanning device, is disclosed, including a body, a scanner coupled with the body, a position sensor coupled to the scanner, a camera configured to take a plurality of images as the scanner moves over a facial feature; and a processor configured to detect a position and a curvature of the facial feature based on the position sensor, combine the plurality of images into a single image, and generate a three-dimensional model of the facial feature.

In some embodiments, the device further includes a light source configured to illuminate the facial feature.

In some embodiments, the camera is a first camera, and the device further comprises a second camera. In some embodiments, the first camera is located on a first side of the scanner, and the second camera is located on a second side opposite the first side of the scanner.

In some embodiments, the device further includes a handle portion coupled to the body. In some embodiments, the device further includes a user interface configured to provide a scanning guide.

In some embodiments, the facial feature is an eyebrow, an eye, a mouth, a nose, a wrinkle, or acne.

In some embodiments, the position sensor is a rolling position sensor. In some embodiments, the position sensor is an accelerometer.

In another aspect, a method including moving a scanning device as described herein over a facial feature, taking a plurality of images of the facial feature with a camera as the scanning device moves over the facial feature, detecting a position of the facial feature with the position sensor as the scanning device moves over the facial feature, combining the plurality of images together, and generating a three-dimensional model of the facial feature is disclosed.

In some embodiments, detecting the position of the facial feature includes detecting a curvature of the facial feature with the position sensor. In some embodiments, the method further includes fabricating a makeup overlay for the facial feature.

In some embodiments, the method includes illuminating the facial feature before taking the plurality of images. In some embodiments, the method includes detecting a lighting of the facial feature; and when the lighting is below a threshold, illuminating the facial feature with a light source on the scanning device. In some embodiments, the method includes detecting a lighting of the facial feature; and when the lighting is below a threshold, issuing an alert to illuminate the facial feature. In some embodiments, the alert is an auditory, visual, or tactile alert.

In some embodiments, the method further includes directing the scanning device to move in a direction over the facial feature. In some embodiments, the method further includes displaying a scanning guide on a user interface of the scanning device. In some embodiments, the scanning guide comprises one or more of the plurality of images of the facial feature and an arrow pointing in a direction a user can move the scanning device. In some embodiments the scanning guide is a graphical representation of the facial feature and an arrow pointing in the direction a user can move the scanning device.

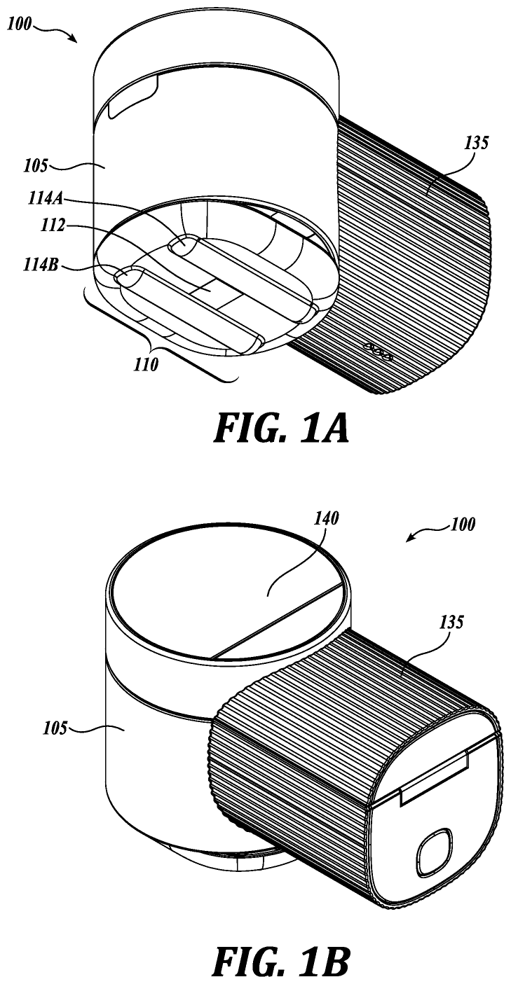

FIG. 1A is a perspective front-side of an example scanning device 100, in accordance with the present technology. In some embodiments, the scanning device 100 includes a body 105, and a handle 135. While scanning device 100 is illustrated with a cylindrical body 105 and a cylindrical handle 135, it should be understood that the scanning device 100 can take any number of forms. In some embodiments, the scanning device 100 does not have a handle 135. In some embodiments, the scanning device 100 includes internal circuitry, including a processor, a battery, and the like. In some embodiments, the scanning device 100 includes a processor configured to detect a position and a curvature of the facial feature based on the position sensor, combine the plurality of images into a single image, and generate a three-dimensional model of the facial feature, as described in detail herein.

In some embodiments, the scanning device 100 is powered through a wired connection, but the scanning device 100 may also be independently powered, such as with a battery or a capacitor. In some embodiments, the scanning device 100 may further include a charging port, configured to power the scanning device 100.

In some embodiments, the body houses the scanner 110. In some embodiments, the scanner 110 is positioned on a front side of the scanning device 100, such as shown in FIG. 1A. In some embodiments, the scanner 110 includes a scanning window 112 and one or more spacers 114A and 114B.

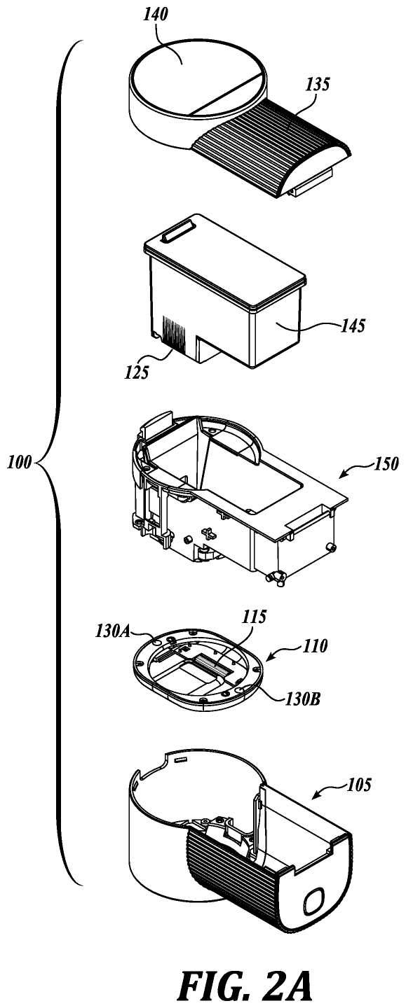

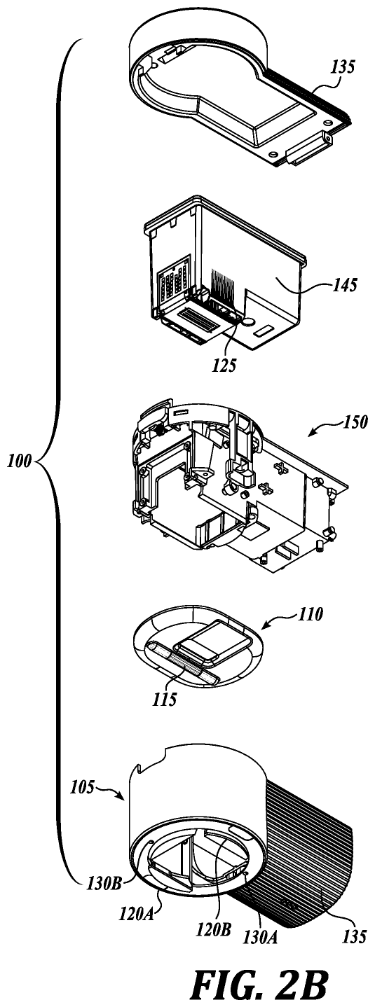

In some embodiments, the scanning window 112 is configured to allow internal scanning components, as shown in FIG. 2B, to visualize the facial feature as described here. In some embodiments, the scanning window 112 is translucent. In some embodiments, the scanning window 112 is rectangular, square, circular, organic, or the like. In some embodiments, the scanning window 112 is in the middle of front side of the body 105. In some embodiments, the scanning window 112 is located between the spacers 114A, 114B.

While two spacers 114A, 114B are illustrated, it should be understood that any number of spacers 114A, 114B may be on the scanner 110. In some embodiments, the spacers 114A, 114B are rounded polygons, such as shown in FIG. 1A, but it should be understood that the spacers 114A, 114B can take any number of forms including spherical, rectangular, and organic. In some embodiments, the spacers 114A, 114B are configured to contact a surface while the scanning device 100 is passed over it, so that an optimal distance between the scanner 110 (or scanning window 112) and the surface is achieved.

In some embodiments, the scanning device 100 includes at least one position sensor coupled to the scanner 110 (as shown and described in FIG. 2A). In some embodiments, the position sensor may be housed inside the body 105, but in some embodiments, the position sensor may be located on the front side of the scanning device with the scanner 110. In some embodiments, the scanning device 100 further includes a camera (as shown and described in FIG. 2A). In some embodiments, the camera is configured to take a plurality of images as the scanner 110 moves over a facial feature. In some embodiments, the facial feature may be an eyebrow, a nose, an eye, a wrinkle, acne, or the like.

In some embodiments, the scanner 110 is a rotatably adjustable body scanner 110. In some embodiments, the scanner 110 is configured to articulate to more accurately scan a surface, such as a body, skin, or hair. In such embodiments, position sensor 115 may be a sensor wheel as described herein. In operation, the position sensor 115 contacts the surface and rolls as the scanner 110 scans the surface. In such embodiments, the scanner 110 is able to take into account the curvature of the surface. In some embodiments, the surface is a body. In some embodiments, the scanner 110 can be adjusted to fit the needs of different body types and scanning environments. In some embodiments, the scanner 110 has an adjustable scanning window 112. In some embodiments, the spacers 114A and 114B may be moved or adjusted to change the size of the scanning window 112. In some embodiments, the scanning window 112 may be concave or convex to capture scans or images of the surface accurately. In some embodiments, the scanner 110 is capable of being articulated, so as to better contact the surface. In some embodiments, the scanner 110 is coupled to the device 100 with a flexible connector. In some embodiments, the flexible connector is a pivot, a hinge, or a joint. In some embodiments, the flexible connector allows the scanner 110 to be articulated. In some embodiments, this allows for more accurate scans of a surface. In some embodiments, this further allows the scanner 110 to determine a curvature of a surface.

FIG. 1B is a perspective back-side of the example scanning device 100 of FIG. 1A, in accordance with the present technology. In some embodiments, the scanning device 100 further includes a user interface 140. Though the user interface 140 is illustrated on the backside of the scanning device 100, in some embodiments, the user interface 140 is a separate component, such as a smartphone or tablet. In some embodiments, the user interface 140 is round, but in other embodiments, the user interface 140 may take any form such as rectangular or oblong. In some embodiments, the user interface 140 includes one or more actuators, such as buttons or keys. In some embodiments, the user interface 140 includes a touch type capacitance button. In some embodiments, the user interface 140 is a touchscreen. In some embodiments, the user interface includes one or more output modules configured to output an alert. In some embodiments, the alert is a sound, vibration, or the like. In some embodiments, the alert includes an indication on how or in what direction to move the scanning device 100.

FIG. 2A is an exploded backside view of an example scanning device 100, in accordance with the present technology and FIG. 2B is an exploded frontside view of the example scanning device of FIG. 2A, in accordance with the present technology.

In some embodiments, the scanning device 100 includes internal scanning components 150, a scanner 110, and a position sensor 115. In some embodiments, the scanning device 100 further includes a printer 145 and a processor 125.

In some embodiments, the internal scanning components 150 are configured to hold the scanner 110 in place. In some embodiments, the internal scanning components 150 are coupled to the scanner 110 and the printer 145.

In some embodiments, the scanner 110 includes a positioning sensor 115 and at least one camera 120A, 120B. In some embodiments, the cameras 120A, 120B are located on the scanner 110 but in some embodiments, the cameras 120A, 120B are located on the body 105. In some embodiments, as the scanner 110 moves across a surface, such as the user's face, the cameras 120A, 120B capture a plurality of images of the surface. In some embodiments, the cameras 120A, 120B take a plurality of images of a facial feature as the scanning device moves over the facial feature. In some embodiments, the scanning device 100 includes two cameras 120A and 120B. In some embodiments, such as illustrated in FIG. 2A, the first camera 120B is located on a first side of the scanning device 100, and the second camera 120B is located on a second side of the scanning device 100, opposite the first side.

In some embodiments, such as shown in FIG. 2B, the scanning device 100 includes one or more light sources 130A, 130B. In some embodiments, the light sources 130A, 130B are LEDs. Though two light sources 130A, 130B are illustrated, any number of light sources 130 may be on the scanning device 100. In some embodiments, the light sources 130A, 130B are positioned on the scanner 110, but in some embodiments, the light sources 130A, 130B are positioned on the front-side of the scanning device 100.

In some embodiments, the scanner 110 includes one or more position sensors 115. While a single position sensor 115 is illustrated in FIG. 2A, it should be understood that any number of position sensors 115 may be used. In some embodiments, at least one position sensor 115 is a rolling position sensor 115, such as a sensor wheel. In such embodiments, the position sensor 115 is configured to roll across the facial feature as the scanner 110 is moved over the facial feature. In this manner, position sensor 115 may detect a position of the facial feature as the scanning device 100 moves over the facial feature. In some embodiments, the position sensor 115 is further configured to detect the curvature of the facial feature or the user's face.

In some embodiments, the scanning device 100 includes a processor 125. In some embodiments, the processor 145 is communicatively coupled to the scanner 110, the position sensor 115, and the camera 120. The processor 125 may be configured to combine the plurality of images from the camera 120 together and generate a three-dimensional model of the facial feature. In some embodiments, the processor is further configured to detect the lighting of the facial feature, and direct one or more light sources 130A, 130B to illuminate the light feature. While a single processor 125 is illustrated, it should be understood that and number of processors may be incorporated into the scanning device 100.

In some embodiments, the processor 125 is further communicatively coupled to the printer 145. In some embodiments, the processor 125 directs the printer 145 to fabricate a makeup overlay, such as a temporary tattoo, or makeup printed in the shape of the facial feature.

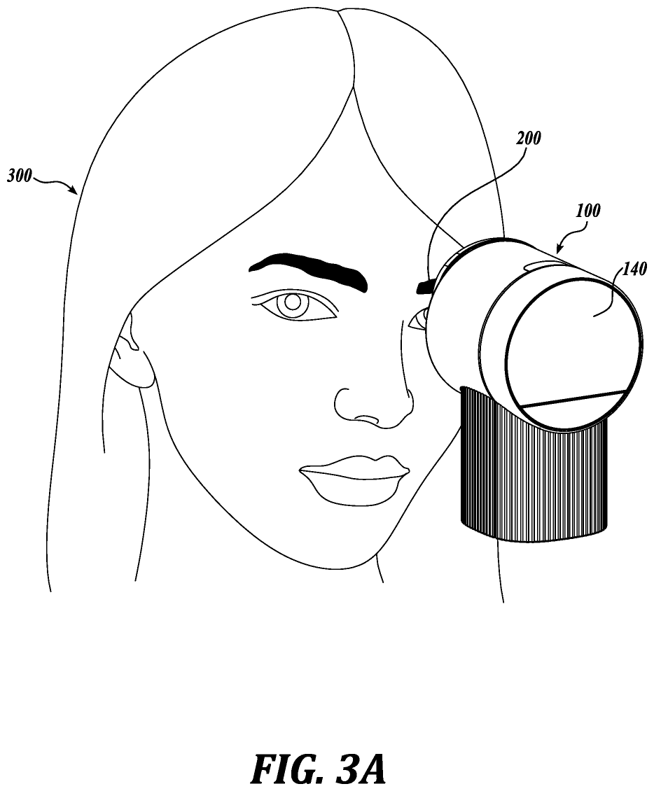

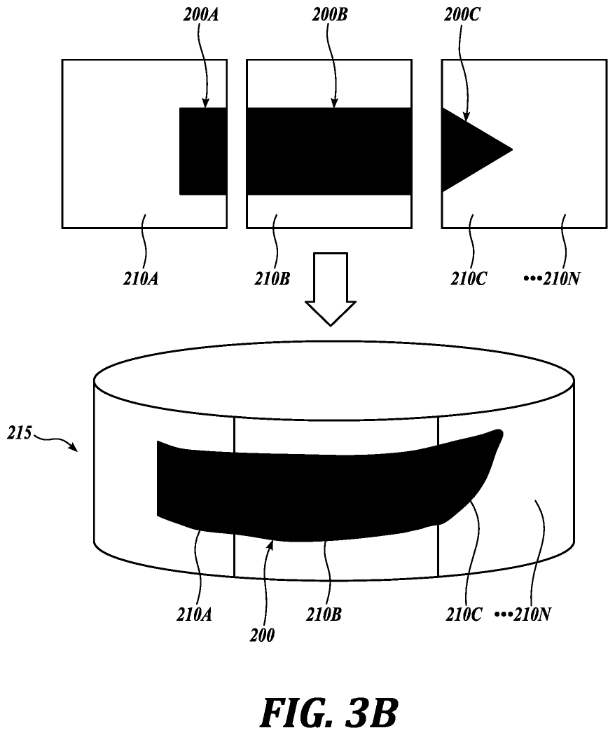

FIG. 3A is an image of an example scanning device 100 in use, in accordance with the present technology. In some embodiments, a user 300 uses the scanning device 100 to generate a three-dimensional model of one or more facial features 200. In some embodiments, the user 300 may be a first person using the device on a second person. In some embodiments, the first person may be a trained user, such as in a store or makeup counter.

In some embodiments, the facial feature 200 is a brow. In some embodiments, the facial feature is a nose, eye, lips, wrinkle, acne, or discoloration of the skin. In some embodiments, the scanning device 100 is configured to recognize any number of types of facial features 200. In this manner, a single scanning device 100 may be used to make a three-dimensional model and/or a makeup overlay of a brow, an eye, a wrinkle, lips, etc.

In operation, a user 300 may hold the scanning device 100 from handle 135 and move the scanner 110 over a surface. In some embodiments, the surface is a face. In some embodiments, the surface is skin or hair. In some embodiments, the surface is a facial feature 200. As the scanning device 100 is moved over the surface, FIG. 3B is an example three-dimensional model generated by a scanning device, in accordance with the present technology.

In some embodiments, the processor, such as processor 125 is further configured to generate a three-dimensional model 215 of the facial feature 200. While the facial feature 200 is illustrated as an eyebrow, it should be understood that the facial feature 200 may be any facial feature, such as a mole, acne, scar, wrinkle, eyelid, eye, lip, etc. In some embodiments, the scanning device 100 is configured to take a plurality of images 210A, 210B, 210C . . . 210N of the facial feature 200. In some embodiments, each image of the plurality of images 210A, 210B, 210C . . . 210N includes at least a portion of the facial feature 200A, 200B, 200C. In some embodiments, the number of images 210A, 210B, 210C . . . 210N depends on how many images are needed to capture the entirety of the facial feature 200.

In some embodiments, the plurality of images 210A, 210B, 210C . . . 210N are compiled to create a three-dimensional model 215 of the facial feature 200. In some embodiments, the position sensor as described herein can detect the depth and curvature of the facial feature 200. In some embodiments, the processor can take the depth and curvature of the facial feature or surface into account when generating the three-dimensional model. In this manner, the three-dimensional model 215 can accurately reflect the curvature of the facial feature 200.

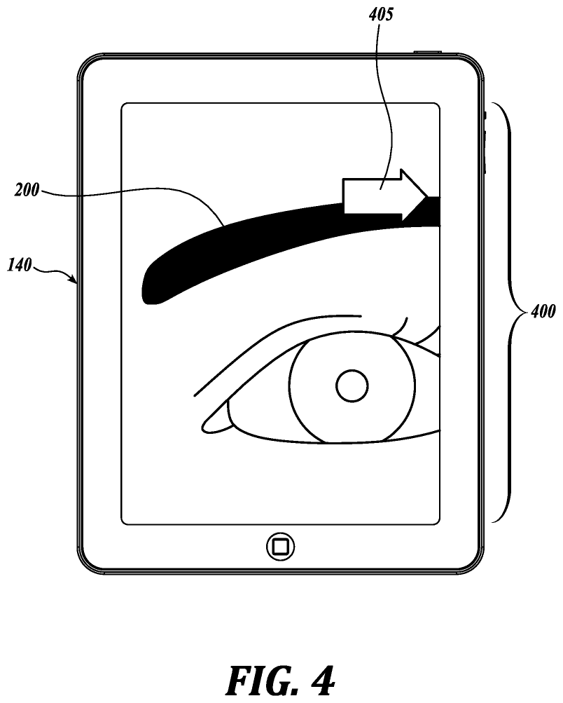

FIG. 4 is a user interface 140 displaying a scanning guide 400, in accordance with the present technology. In some embodiments, the user interface 140 is configured to display a scanning guide 400 in order to direct a user to properly use the scanning device 100. In some embodiments, the scanning guide 400 includes one or more of the plurality of images as described herein of the facial feature 200 and an arrow 405 pointing in a direction a user can move the scanning device. In some embodiments, the scanning guide 400 includes a graphical representation of the facial feature 200 and an arrow 405 pointing in the direction a user can move the scanning device.

In some embodiments, the user interface 140 displays a current view of the camera on the scanning device. In some embodiments, as the user moves the scanning device over the surface, an image captured by the camera is displayed.

In some embodiments, the scanning guide 400 further includes one or more alerts to direct the user to move the scanning device. In some embodiments, the alerts are visual alerts, such as arrow 405, auditory alerts, such as a chime or alarm, or tactile alerts such as vibrations.

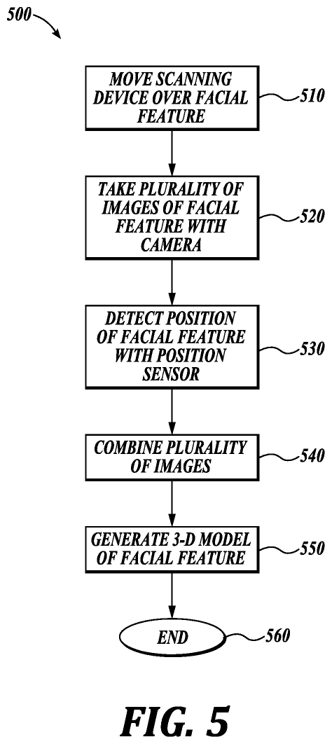

FIG. 5 is an example method 500 of using a scanning device, in accordance with the present technology.

Method 500 begins in block 510. In block 510, a scanning device (such as scanning device 100 of FIG. 1) is moved over a facial feature (such as facial feature 200). The method 500 then proceeds to block 520.

In block 520, a plurality of images of the facial feature are taken with a camera. In some embodiments, the camera is located on the scanning device. The method 500 then proceeds to block 530.

In block 530, a position sensor detects a position of the facial feature. In some embodiments, the position of the facial feature includes a curvature of the facial feature. In some embodiments, the position of the facial feature includes a depth of a facial feature. In some embodiments, the position sensor is a rolling position sensor. In this manner, the position sensor may detect the position of the facial feature as the position sensor rolls over the facial feature. The method 500 then proceeds to block 540.

In block 540, the plurality of images taken by the camera are combined. In some embodiments, combining the images includes stitching together the images based on the position as indicated by the position sensor. The method 500 then proceeds to block 550.

In block 550, a three-dimensional model of the facial feature is generated. In some embodiments, the three-dimensional model takes the position from the position sensor into account. In some embodiments, the three-dimensional model takes the curvature and/or depth of the facial feature into account. In some embodiments, the three-dimensional model is then used to fabricate a makeup overlay, as described herein. The method 500 then proceeds to block 560.

In block 560, the method ends.

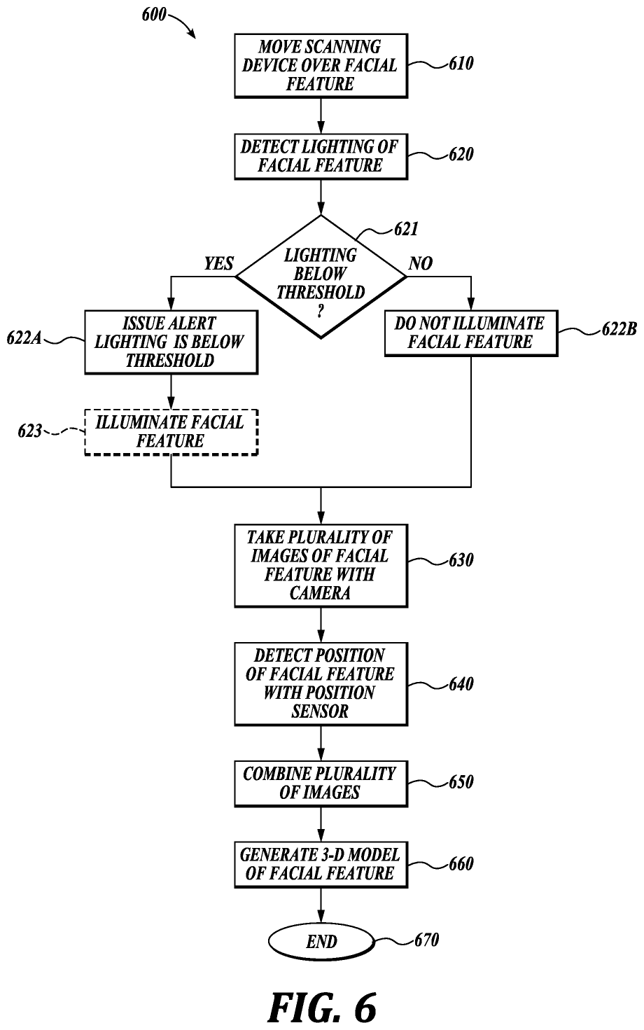

FIG. 6 is an example method 600 of using a scanning device having a light source, in accordance with the present technology.

Method 600 begins in block 610. In block 610, a scanning device (such as scanning device 100 of FIG. 1) is moved over a facial feature (such as facial feature 200). The method then proceeds to block 620.

In block 620, the lighting of the facial feature is detected. In some embodiments, the scanning device detects the lighting of the facial feature to determine if the images will be of sufficient quality to generate the three dimensional model. The method then proceeds to decision block 621.

In decision block 621, a determination is made regarding if the lighting is below a threshold. In some embodiments, the threshold may be set by the user. In some embodiments, the threshold is hard coded into the device. If the lighting is below the threshold, the method proceeds to block 622A.

In block 622A, the device issues an alert that the lighting is below a threshold. In some embodiments, the alert may be a visual alert, an auditory alert, or a tactile alert. The method then proceeds to block 623.

Optionally, the device may then illuminate the facial feature. In some embodiments, the facial feature is illuminated with one or more light sources located on the scanning device. In some embodiments, the one or more light sources are LEDs. The method then proceeds to block 630.

Returning to block 621, if the lighting is not below the threshold, the method proceeds to block 622B.

In block 622B, the device does not illuminate the facial feature. The method then proceeds to block 630.

In block 630, a plurality of images of the facial feature are taken with a camera. In some embodiments, the camera is located on the scanning device. The method 600 then proceeds to block 640.

In block 640, a position sensor detects a position of the facial feature. In some embodiments, the position of the facial feature includes a curvature of the facial feature. In some embodiments, the position of the facial feature includes a depth of a facial feature. In some embodiments, the position sensor is a rolling position sensor. In this manner, the position sensor may detect the position of the facial feature as the position sensor rolls over the facial feature. The method 600 then proceeds to block 650.

In block 650, the plurality of images taken by the camera are combined. In some embodiments, combining the images includes stitching together the images based on the position as indicated by the position sensor. The method 600 then proceeds to block 660.

In block 660, a three-dimensional model of the facial feature is generated. In some embodiments, the three-dimensional model takes the position from the position sensor into account. In some embodiments, the three-dimensional model takes the curvature and/or depth of the facial feature into account. In some embodiments, the three-dimensional model is then used to fabricate a makeup overlay, as described herein. The method 600 then proceeds to block 670.

In block 670, the method 600 ends.

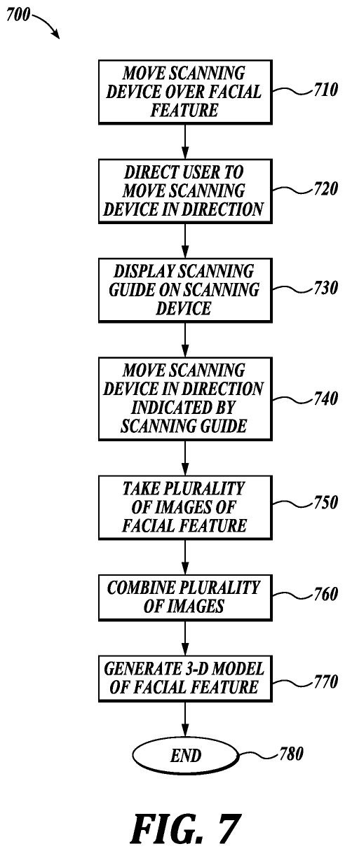

FIG. 7 is an example method 700 of using a scanning device with a scanning guide, in accordance with the present technology.

Method 700 begins in block 710. In block 710, a scanning device (such as scanning device 100 of FIG. 1) is moved over a facial feature (such as facial feature 200). The method 700 then proceeds to block 720.

In block 720, the user is directed to move the scanning device in a direction. In some embodiments, the user is directed to move the scanning device by the device itself. In some embodiments, the direction may be an alert, such as a tactile alert, visual alert, or auditory alert. The method 700 then proceeds to block 730.

In block 730, the scanning device displays a scanning guide (such as scanning guide 400). In some embodiments, the scanning guide is displayed on a user interface of the scanning device. In some embodiments, the scanning guide may show an image as it is taken by a camera on the scanning device, and an arrow indicating the direction the user should move the scanning device to fully capture a facial feature. In some embodiments, the scanning guide may be a graphical representation of the facial feature and include an arrow indicating the direction the user should move the device. The method 700 then proceeds to block 740.

In block 740, the scanning device is moved in the direction indicated by the scanning guide. The method 700 then proceeds to block 750.

In block 750, a plurality of images of the facial feature are taken with a camera. In some embodiments, the camera is located on the scanning device. The method 700 then proceeds to block 760.

In block 760, the plurality of images taken by the camera are combined. In some embodiments, combining the images includes stitching together the images based on the position as indicated by the position sensor. The method 700 then proceeds to block 770.

In block 770, a three-dimensional model of the facial feature is generated. In some embodiments, the three-dimensional model takes the position from the position sensor into account. In some embodiments, the three-dimensional model takes the curvature and/or depth of the facial feature into account. In some embodiments, the three-dimensional model is then used to fabricate a makeup overlay, as described herein. The method 700 then proceeds to block 780.

In block 780, the method 700 ends.

It should be understood that the methods 500, 600, and 700 are merely representative and may include additional steps. Further, each step of methods 500, 600, and 700 may be performed in any order, or even be omitted.

While illustrative embodiments have been illustrated and described, it will be appreciated that various changes can be made therein without departing from the spirit and scope of the invention.

Embodiments disclosed herein may utilize circuitry in order to implement technologies and methodologies described herein, operatively connect two or more components, generate information, determine operation conditions, control an appliance, device, or method, and/or the like. Circuitry of any type can be used. In an embodiment, circuitry includes, among other things, one or more computing devices such as a processor (e.g., a microprocessor), a central processing unit (CPU), a digital signal processor (DSP), an application-specific integrated circuit (ASIC), a field-programmable gate array (FPGA), or the like, or any combinations thereof, and can include discrete digital or analog circuit elements or electronics, or combinations thereof.

In an embodiment, circuitry includes one or more ASICs having a plurality of predefined logic components. In an embodiment, circuitry includes one or more FPGA having a plurality of programmable logic components. In an embodiment, circuitry includes hardware circuit implementations (e.g., implementations in analog circuitry, implementations in digital circuitry, and the like, and combinations thereof). In an embodiment, circuitry includes combinations of circuits and computer program products having software or firmware instructions stored on one or more computer readable memories that work together to cause a device to perform one or more methodologies or technologies described herein. In an embodiment, circuitry includes circuits, such as, for example, microprocessors or portions of microprocessor, that require software, firmware, and the like for operation. In an embodiment, circuitry includes an implementation comprising one or more processors or portions thereof and accompanying software, firmware, hardware, and the like. In an embodiment, circuitry includes a baseband integrated circuit or applications processor integrated circuit or a similar integrated circuit in a server, a cellular network device, other network device, or other computing device. In an embodiment, circuitry includes one or more remotely located components. In an embodiment, remotely located components are operatively connected via wireless communication. In an embodiment, remotely located components are operatively connected via one or more receivers, transmitters, transceivers, or the like.

An embodiment includes one or more data stores that, for example, store instructions or data. Non-limiting examples of one or more data stores include volatile memory (e.g., Random Access memory (RAM), Dynamic Random Access memory (DRAM), or the like), non-volatile memory (e.g., Read-Only memory (ROM), Electrically Erasable Programmable Read-Only memory (EEPROM), Compact Disc Read-Only memory (CD-ROM), or the like), persistent memory, or the like. Further non-limiting examples of one or more data stores include Erasable Programmable Read-Only memory (EPROM), flash memory, or the like. The one or more data stores can be connected to, for example, one or more computing devices by one or more instructions, data, or power buses.

In an embodiment, circuitry includes one or more computer-readable media drives, interface sockets, Universal Serial Bus (USB) ports, memory card slots, or the like, and one or more input/output components such as, for example, a graphical user interface, a display, a keyboard, a keypad, a trackball, a joystick, a touch-screen, a mouse, a switch, a dial, or the like, and any other peripheral device. In an embodiment, circuitry includes one or more user input/output components that are operatively connected to at least one computing device to control (electrical, electromechanical, software-implemented, firmware-implemented, or other control, or combinations thereof) one or more aspects of the embodiment.

In an embodiment, circuitry includes a computer-readable media drive or memory slot configured to accept signal-bearing medium (e.g., computer-readable memory media, computer-readable recording media, or the like). In an embodiment, a program for causing a system to execute any of the disclosed methods can be stored on, for example, a computer-readable recording medium (CRMM), a signal-bearing medium, or the like. Non-limiting examples of signal-bearing media include a recordable type medium such as any form of flash memory, magnetic tape, floppy disk, a hard disk drive, a Compact Disc (CD), a Digital Video Disk (DVD), Blu-Ray Disc, a digital tape, a computer memory, or the like, as well as transmission type medium such as a digital and/or an analog communication medium (e.g., a fiber optic cable, a waveguide, a wired communications link, a wireless communication link (e.g., transmitter, receiver, transceiver, transmission logic, reception logic, etc.). Further non-limiting examples of signal-bearing media include, but are not limited to, DVD-ROM, DVD-RAM, DVD+RW, DVD-RW, DVD-R, DVD+R, CD-ROM, Super Audio CD, CD-R, CD+R, CD+RW, CD-RW, Video Compact Discs, Super Video Discs, flash memory, magnetic tape, magneto-optic disk, MINIDISC, non-volatile memory card, EEPROM, optical disk, optical storage, RAM, ROM, system memory, web server, or the like.

The detailed description set forth above in connection with the appended drawings, where like numerals reference like elements, are intended as a description of various embodiments of the present disclosure and are not intended to represent the only embodiments. Each embodiment described in this disclosure is provided merely as an example or illustration and should not be construed as preferred or advantageous over other embodiments. The illustrative examples provided herein are not intended to be exhaustive or to limit the disclosure to the precise forms disclosed. Similarly, any steps described herein may be interchangeable with other steps, or combinations of steps, in order to achieve the same or substantially similar result. Generally, the embodiments disclosed herein are non-limiting, and the inventors contemplate that other embodiments within the scope of this disclosure may include structures and functionalities from more than one specific embodiment shown in the figures and described in the specification.

In the foregoing description, specific details are set forth to provide a thorough understanding of exemplary embodiments of the present disclosure. It will be apparent to one skilled in the art, however, that the embodiments disclosed herein may be practiced without embodying all the specific details. In some instances, well-known process steps have not been described in detail in order not to unnecessarily obscure various aspects of the present disclosure. Further, it will be appreciated that embodiments of the present disclosure may employ any combination of features described herein.

The present application may include references to directions, such as “vertical,” “horizontal,” “front,” “rear,” “left,” “right,” “top,” and “bottom,” etc. These references, and other similar references in the present application, are intended to assist in helping describe and understand the particular embodiment (such as when the embodiment is positioned for use) and are not intended to limit the present disclosure to these directions or locations.

The present application may also reference quantities and numbers. Unless specifically stated, such quantities and numbers are not to be considered restrictive, but exemplary of the possible quantities or numbers associated with the present application. Also in this regard, the present application may use the term “plurality” to reference a quantity or number. In this regard, the term “plurality” is meant to be any number that is more than one, for example, two, three, four, five, etc. The term “about,” “approximately,” etc., means plus or minus 5% of the stated value. The term “based upon” means “based at least partially upon.”

The principles, representative embodiments, and modes of operation of the present disclosure have been described in the foregoing description. However, aspects of the present disclosure, which are intended to be protected, are not to be construed as limited to the particular embodiments disclosed. Further, the embodiments described herein are to be regarded as illustrative rather than restrictive. It will be appreciated that variations and changes may be made by others, and equivalents employed, without departing from the spirit of the present disclosure. Accordingly, it is expressly intended that all such variations, changes, and equivalents fall within the spirit and scope of the present disclosure as claimed.

1. A scanning device, comprising:
a body;
a scanner coupled with the body;
a position sensor coupled to the scanner;
a camera configured to take a plurality of images as the scanner moves over a facial feature; and
a processor configured to:
detect a position and a curvature of the facial feature based on the position sensor;
combine the plurality of images into a single image; and
generate a three-dimensional model of the facial feature.

2. The device of claim 1, further comprising a light source configured to illuminate the facial feature.

3. The device of claim 1, wherein the camera is a first camera, and the device further comprises a second camera.

4. The device of claim 3, wherein the first camera is located on a first side of the scanner, and the second camera is located on a second side opposite the first side of the scanner component.

5. The device of claim 1, further comprising a user interface configured to provide a scanning guide.

6. The device of claim 1, wherein the facial feature is an eyebrow, an eye, a mouth, a nose, a wrinkle, or acne.

7. The device of claim 1, wherein the position sensor is configured to contact a surface as the position sensor rolls over the surface and measure a curvature of the surface.

8. The device of claim 1, wherein the scanner the scanner is coupled with the device with a flexible connector.

9. The device of claim 8, wherein the flexible connector is a pivot, a hinge, or a joint.

10. The device of claim 1, wherein the position sensor is an accelerometer.

11. A method comprising:
moving the scanning device of claim 1 over a facial feature;
capturing a plurality of images of the facial feature with the camera as the scanning device moves over the facial feature;
detecting a position of the facial feature with the position sensor as the scanning device moves over the facial feature;
combining the plurality of images together; and
generating a three-dimensional model of the facial feature.

12. The method of claim 11, wherein detecting the position of the facial feature comprises detecting a curvature of the facial feature with the position sensor.

13. The method of claim 11, wherein the method further comprises fabricating a makeup overlay for the facial feature.

14. The method of claim 11, further comprising:
illuminating the facial feature before taking the plurality of images.

15. The method of claim 14, further comprising:
detecting a lighting of the facial feature; and
when the lighting is below a threshold, illuminating the facial feature with a light source on the scanning device.

16. The method of claim 14, further comprising:
detecting a lighting of the facial feature; and
when the lighting is below a threshold, issuing an alert to illuminate the facial feature.

17. The method of claim 14, further comprising:
directing the scanning device to move in a direction over the facial feature.

18. The method of claim 17, further comprising:
displaying a scanning guide on a user interface of the scanning device.

19. The method of claim 18, wherein the scanning guide comprises one or more of the plurality of images of the facial feature and an arrow pointing in a direction a user can move the scanning device.

20. The method of claim 18, wherein the scanning guide comprises a graphical representation of the facial feature and an arrow pointing in the direction a user can move the scanning device.
